SEQUENCE LISTING (6 sequences)

SEQ ID NO: 1 - DNA, 20 bp, Artificial Sequence
  artificially synthesized
atgtccgaagcaaacatcac

SEQ ID NO: 2 - DNA, 20 bp, Artificial Sequence
  artificially synthesized
taatgtccaggaagtaggtg

SEQ ID NO: 3 - DNA, 20 bp, Artificial Sequence
  artificially synthesized
aggccggtgctgagtatgtc

SEQ ID NO: 4 - DNA, 20 bp, Artificial Sequence
  artificially synthesized
tgcctgcttcaccaccttct

SEQ ID NO: 5 - DNA, 27 bp, Artificial Sequence
  artificially synthesized
ggggtaccttccagctgtcaaaatctc

SEQ ID NO: 6 - DNA, 25 bp, Artificial Sequence
  artificially synthesized
gaagatctcgccaggtactcacctg

(19) United States
(12) Patent Application Publication
(10) Pub. No.: US 2014/0100286 A1
(43) Pub. Date: Apr. 10, 2014

(54) COMPOSITION FOR PREVENTING OR TREATING INFLAMMATORY DISEASES COMPRISING VERATRIC ACID AS EFFECTIVE COMPONENT

(71) Applicant: Pukyong National University Industry-University Cooperation Foundation, Busan (KR)

(72) Inventors: Gun-Do KIM, Busan (KP); Gyun-Pyung SHIN, Gyeonggi-do (KR); Woo-Suk CHOI, Busan (KR); Sang-Yeol LEE, Jinju (KR); Woe-Yeon KIM, Jinju (KR); Nam-Gyu PARK, Busan (KR)

(21) Appl. No.: 13/929,523

(22) Filed: Jun. 27, 2013

(30) Foreign Application Priority Data
Oct. 10, 2012 (KR) 10-2012-0112595

(51) Int. Cl. A61K 31/192; A23L 1/30

(52) U.S. Cl. CPC A61K 31/192; A23L 1/30
USPC 514/568; 562/476

(57) ABSTRACT

There is provided a composition including a veratric acid for preventing or treating inflammatory diseases. The veratric acid of the present invention has an excellent effect of inhibiting production of nitric oxide, an excellent activity of inhibiting production of IL-1β as a pro-inflammatory cytokine, and an excellent effect of inhibiting phosphorylation of MAPK, NF-κB, GSK-3β, and c-Raf in a macrophage cell stimulated by a stimulation factor that causes an inflammatory reaction. Thus, it can be used for development of medicines for treating inflammatory diseases which can be caused by excessive inflammatory reactions. Further, the veratric acid of the present invention does not induce toxicity to cells and is stable in the body. Thus, it can be used as a material of a health functional food capable of preventing or improving inflammatory diseases.

### CROSS-REFERENCE TO RELATED APPLICATIONS

This application claims the priority of Korean Patent Application No. 2012-0112595 filed on Oct. 10, 2012, in the Korean Intellectual Property Office, the disclosure of which is incorporated herein by reference.

### BACKGROUND OF THE INVENTION

1. Field of the Invention

The present invention relates to a novel use of a veratric acid capable of effectively preventing and treating inflammatory diseases.

2. Description of the Related Art

Inflammatory disorders are one of the most important health problems in the world. Inflammation is in general a localized protective response of body tissues to invasion of a host by foreign materials or injurious stimuli. Causes of inflammation can be infectious causes such as bacteria, viruses, and parasites; physical causes such as burns or radiation exposure; chemicals such as toxins, drugs or industrial agents; immunological reactions such as allergies and autoimmune reactions; or conditions associated with oxidative stress.

Inflammation is characterized by pain, redness, swelling, heat, and eventual loss of function of an infected area. These symptoms are results of a series of complex interactions occurring between cells of an immune system. A response of the cells results in an interacting network of several groups of inflammatory mediators: proteins (for example, a cytokine, an enzyme (e.g. protease, peroxidase), major basic proteins, adhesion molecules (ICAM, VCAM), lipid mediators (for example, eicosanoid, prostaglandin, leukotriene, a platelet activating factor (PAF)), and reactive oxygen species (for example, hydroperoxide, superoxide anion (O2-), nitric oxide (NO), etc.). However, many of those mediators of inflammation are also regulators of normal cellular activities. Therefore, deficiencies of inflammatory reactions lead to an uncontrolled and compromised host (i.e. infection), and thus, chronic inflammation leads to inflammatory diseases mediated in part by excessive production of several of the above-mentioned mediators.

In particular, autoimmune diseases as one of inflammatory diseases are characterized by a spontaneous response to an attack of an immune system of an individual against its own organs. Such a response is caused by recognition of an auto-antigen by T lymphocytes, which results in a secretory immune reaction (production of an auto-antigen) and a cellular immune reaction (increase in cytotoxic activity of a lymphocyte and a macrophage cell). The autoimmune diseases may include: rheumatic diseases, psoriasis, systemic dermatomyositis, multiple sclerosis, lupus erythematosus, or deterioration of immune reactions to antigens, i.e. asthma and allergies to drugs or foods. All of these diseases are restrictive and chronic diseases and can be fatal diseases in certain circumstances. Until the present, any effective treatment method for the above-mentioned diseases has not been suggested. Therefore, a drug, medicine, or a medium capable of alleviating or relieving a progressive disease can be an important means for solving a health problem of a patient.

There has been made a lot of effort to find appropriate drugs and methods by searching treatment methods for autoimmune diseases. Today, treatments for autoimmune diseases are mainly based on a use of immunosuppressive drugs such as glucocorticoids, calcineurin inhibitors, and antiproliferatives-antimetabolites. However, such pharmacological treatments act on various targets and thus may deteriorate overall immunity function. Otherwise, if these pharmacological treatments are used for a long time, various cytotoxic activities may suppress an immune system in a non-specific manner, so that a patient may be exposed to a risk of infectious diseases or cancers. The calcineurin and glucocorticoid may have other problems caused by their nephrotoxicity and diabetogenic properties, and thus, they are limited in use for some clinical symptoms (for example, renal insufficiency, diabetes, etc.).

For this reason, patients with immune diseases including autoimmune disease or inflammatory diseases have special interests in treatment considered as “natural” treatment with mild anti-inflammatory effects and without major side effects, which can be used for disease prevention and as adjuvant treatment, and a lot of researchers have increased interests in development of nature-originated medicines.

Recently, there have been made some researches on natural substances to develop stable medicines with fewer side effects. As conventional technologies, Korean Patent No. 668,067 describes that phenylbutenoid derivatives isolated from ginger has excellent anti-inflammatory activity and Korean Patent No. 396,526 describes that xanthorrhizol isolated from Curcuma xanthorrhiza Roxb. has an anti-inflammatory activity and thus can be used for treating inflammatory diseases.

Meanwhile, the present inventors first established that a veratric acid contained in mushroom has excellent anti-inflammatory activity. In particular, the present inventors have completed the present invention by finding that in a macrophage cell stimulated by lipopolysaccharide (LPS), the veratric acid has an activity of inhibiting production of interleukin-1β (IL-1β) as an inflammation-inducing factor, an activity of inhibiting inducible nitric oxide synthase (iNOS) as a nitric oxide synthase involved in inhibition of production of nitric oxide (NO) and production of nitric oxide, inhibitory kappa B-alpha (IκBα), NF-κB, interferon-γ (IFN-γ), STAT-1, glycogen synthase kinase-3β (GSK-3β), and RAF proto-oncogene serine/threonine-protein kinase (c-Raf), and an activity of inhibiting inflammation through a mechanism for inhibiting activities of mechanisms of extracellular signal-regulated kinase (ERK), c-Jun N-terminal kinase (JNK), and p38 as members of the MAPK family.

### SUMMARY OF THE INVENTION

An aspect of the present invention provides a pharmaceutical composition comprising a veratric acid as an effective component for preventing or treating inflammatory diseases.

An aspect of the present invention also provides a health functional food comprising a veratric acid as an effective component for preventing or improving inflammatory diseases.

An aspect of the present invention also provides an anti-inflammatory drug comprising the pharmaceutical composition according to the present invention as an effective component.

Further, an aspect of the present invention provides a method for treating or preventing an inflammatory disease comprising administering to a subject in need thereof a therapeutically effective amount of a veratric acid.

According to an aspect of the present invention, there is provided a pharmaceutical composition comprising a veratric acid represented by the following Chemical Formula 1 as an effective component for preventing or treating an inflammatory disease.

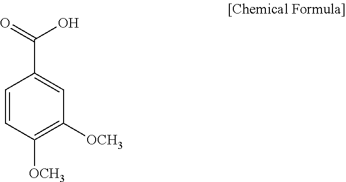 [SMILES: COc1ccc(C(=O)O)cc1OC]

According to an example of the present invention, the veratric acid can inhibit production or expression of nitric oxide, inducible nitric oxide synthase (iNOS), and interferon-γ (INF-γ); and reduce or inhibit phosphorylation of NF-κB, MAP Kinase (MAPK), STAT-1, GSK-3β, and c-Raf.

According to an example of the present invention, the veratric acid may be contained at a concentration of 0.1 to 200 μM in the composition.

According to an example of the present invention, the inflammatory disease may be any one selected from the group consisting of inflammatory bowel disease, peritonitis, osteomyelitis, cellulites, pancreatitis, trauma-induced shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, infectious arthropathy, post-infectious arthritis, gonococcal arthritis, tuberculous arthritis, viral arthritis, fungal arthritis, syphilitic arthritis, Lyme disease, arthritis related to “vasculitis syndrome”, polyarteritis nodosa, hypersensitivity vasculitis, Lou Gehrig's granulomatosis, polymyalgia rheumatica, joint cell arteritis, calcium pyrophosphate deposition arthropathy, pseudogout, non-articular rheumatism, bursitis, tendovaginitis, epicondylitis (tennis elbow), neuropathic joint disease, hemarthrosis, Henoch-Schδnlein purpura, hypertrophic osteoarthropathy, multicentric reticulohistiocytoma, scoliosis, hemochromatosis, hemoglobinopathy, hyperlipoproteinemia, hypogammaglobulinemia, familial Mediterranean fever, Behat's disease, systemic lupus erythematosus, relapsing fever, multiple sclerosis, septicemia, septic shock, acute respiratory distress syndrome, multiorgan dysfunction syndrome, chronic obstructive pulmonary disease, rheumatoid arthritis, acute lung injury, broncho-pulmonary dysplasia, and inflammatory skin disease.

Further, according to another aspect of the present invention, there is provided an anti-inflammatory drug comprising a pharmaceutical composition for preventing or treating an inflammatory disease according to the present invention.

Furthermore, according to still another aspect of the present invention, there is provided a health functional food comprising a veratric acid as an effective component for preventing or improving an inflammatory disease.

According to an example of the present invention, the veratric acid can inhibit production or expression of nitric oxide, inducible nitric oxide synthase (iNOS), and interferon-γ (INF-γ); and reduce or inhibit phosphorylation of NF-κB, MAP Kinase (MAPK), STAT-1, GSK-3β, and c-Raf.

According to an example of the present invention, the inflammatory disease may be any one selected from the group consisting of inflammatory bowel disease, peritonitis, osteomyelitis, cellulites, pancreatitis, trauma-induced shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, infectious arthropathy, post-infectious arthritis, gonococcal arthritis, tuberculous arthritis, viral arthritis, fungal arthritis, syphilitic arthritis, Lyme disease, arthritis related to “vasculitis syndrome”, polyarteritis nodosa, hypersensitivity vasculitis, Lou Gehrig's granulomatosis, polymyalgia rheumatica, joint cell arteritis, calcium pyrophosphate deposition arthropathy, pseudogout, non-articular rheumatism, bursitis, tendovaginitis, epicondylitis (tennis elbow), neuropathic joint disease, hemarthrosis, Henoch-Schδnlein purpura, hypertrophic osteoarthropathy, multicentric reticulohistiocytoma, scoliosis, hemochromatosis, hemoglobinopathy, hyperlipoproteinemia, hypogammaglobulinemia, familial Mediterranean fever, Behat's disease, systemic lupus erythematosus, relapsing fever, multiple sclerosis, septicemia, septic shock, acute respiratory distress syndrome, multiorgan dysfunction syndrome, chronic obstructive pulmonary disease, rheumatoid arthritis, acute lung injury, broncho-pulmonary dysplasia, and inflammatory skin disease.

Furthermore, according to still another aspect of the present invention, there is provided a method for treating or preventing an inflammatory disease comprising administering to a subject in need thereof a therapeutically effective amount of a veratric acid.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 shows a structure of a veratric acid according to the present invention;

FIG. 2 shows a result of cell viability assay of macrophage (RAW264.7) cells, HaCaT cells, and HEK293 cells treated at different concentrations of a veratric acid;

FIG. 3A shows a measurement result for comparison on whether nitric oxide (NO) is produced or not in macrophage (RAW264.7) cells treated with a veratric acid and LPS independently;

FIG. 3B shows a measurement result of inhibition of nitric oxide production in LPS-stimulated macrophage (RAW264.7) cells treated at different concentrations of a veratric acid;

FIGS. 4A and 4B show measurement results of expression of genes and proteins of inducible nitric oxide synthase (iNOS) in LPS-stimulated macrophage (RAW264.7) cells treated at different concentrations of a veratric acid;

FIG. 5 shows a measurement result of expression of genes and proteins of cyclooxygenase-2 (COX-2) in LPS-stimulated macrophage (RAW264.7) cells treated at different concentrations of a veratric acid;

FIGS. 6A and 6B show measurement results of expression of proteins of TNF-α, IL-1β, IL-6, and IFN-γ in LPS-stimulated macrophage (RAW264.7) cells treated at different concentrations of a veratric acid;

FIGS. 7A and 7B show measurement results of expression of proteins of STAT-1 in LPS-stimulated macrophage (RAW264.7) cells treated at different concentrations of a veratric acid;

FIGS. 8A and 8B show measurement results of expression of proteins of NF-κB and IκBα in LPS-stimulated macrophage (RAW264.7) cells treated at different concentrations of a veratric acid;

FIGS. 8C and 8D show measurement results of intracellular distribution patterns of NF-κB in LPS-stimulated macrophage (RAW264.7) cells treated at different concentrations of a veratric acid by using an immunofluorescent microscope;

FIGS. 9A and 9B show measurement results of expression of proteins of JNK, ERK1/2, and p38 in LPS-stimulated macrophage (RAW264.7) cells treated at different concentrations of a veratric acid;

FIGS. 10A and 10B show measurement results of expression of proteins of GSK-3β in LPS-stimulated macrophage (RAW264.7) cells treated at different concentrations of a veratric acid; and

FIGS. 11A and 11B show measurement results of expression of proteins of c-Raf in LPS-stimulated macrophage (RAW264.7) cells treated at different concentrations of a veratric acid.

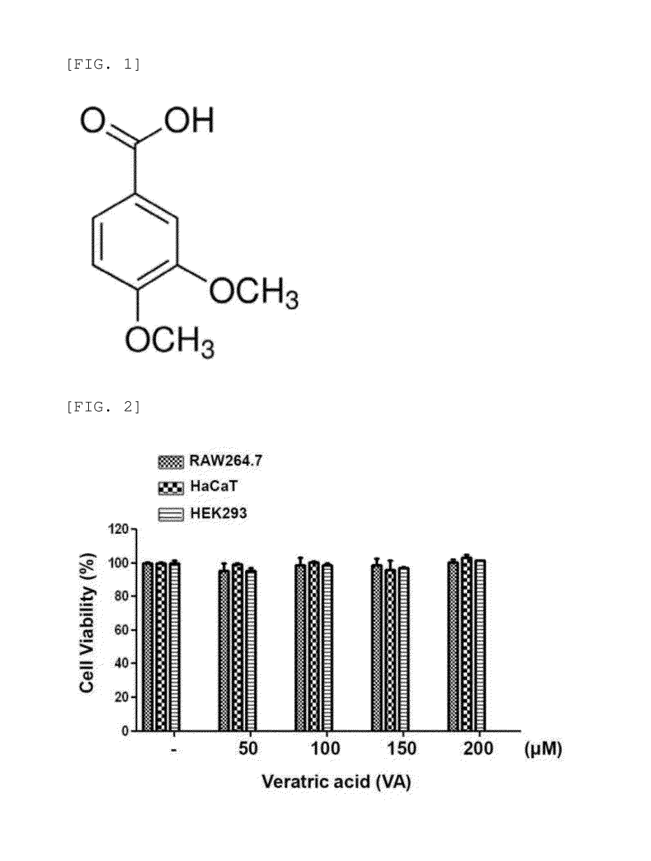

### DETAILED DESCRIPTION OF THE PREFERRED EMBODIMENT

Exemplary embodiments of the present invention will now be described in detail with reference to the accompanying drawings.

The present invention is characterized by providing a novel use of a veratric acid compound as a new medicine for treating inflammatory diseases with excellent anti-inflammatory activity without causing intracellular toxicity.

To be specific, according to the present invention, the veratric acid may be a compound having a structural formula represented by the following Chemical Formula.

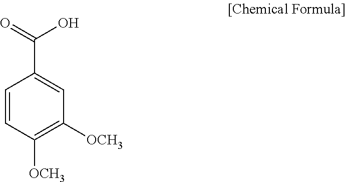 [SMILES: COc1ccc(C(=O)O)cc1OC]

Further, according to the present invention, the veratric acid compound may be used in the form of salt and preferably in the form of pharmaceutically acceptable salt. Preferably, as the salt, there may be used an acid addition salt formed with a pharmaceutically acceptable free acid. As the free acid, an organic acid and an inorganic acid may be used. The organic acid may include, but is not limited to, an citric acid, an acetic acid, a lactic acid, a tartaric acid, a maleic acid, a fumaric acid, a formic acid, a propionic acid, an oxalic acid, a trifluoroacetic acid, a benzoic acid, a gluconic acid, a meta sulfonic acid, a glycolic acid, a succinic acid, a 4-toluenesulfonic acid, a glutamic acid, and an aspartic acid. Further, the inorganic acid may include, but is not limited to, a hydrochloric acid, a bromic acid, a sulfuric acid, and a phosphoric acid.

Further, the veratric acid compound as an effective component for treating inflammatory diseases according to the present invention can be separated from a natural substance or can be produced by a chemical synthesis method publicly known in the art.

If the compound is separated from a natural substance, the compound can be acquired from a plant or a part of a plant by using a conventional method for extracting and separating a material. In order to acquire a target extract from a plant including stems, roots, and leaves, the plant is appropriately dehydrated and macerated or just dehydrated. The target extract may be purified by using a purifying method publicly known in the art. Otherwise, if the compound is produced by a chemical synthesis method, a synthetic compound corresponding to the compound or their derivatives may be typically purchased or may be chemically produced by using a publicly-known synthesis method.

Meanwhile, the veratric acid compound of the present invention has an anti-inflammatory activity of inhibiting inflammatory mediators.

That is, according to an example of the present invention, it was observed that a veratric acid effectively inhibited an inflammatory reaction caused by LPS-treated macrophage (RAW264.7) cells. To be specific, it was observed that the veratric acid effectively inhibited production of nitric oxide and expression of iNOS caused by the LPS and also inhibited production of IFN-γ and IL-1β as pro-inflammatory cytokines.

Further, according to another example of the present invention, it was found that the veratric acid compound of the present invention inhibited phosphorylation of STAT-1, NF-κB, GSK-3β, and c-Raf so as to inhibit production of nitric oxide.

The lipopolysaccharide (LPS) as an endotoxic substance induces production of an inflammation-inducing factor and promotes production of pro-inflammatory cytokines that cause inflammatory reactions. That is, if external stimuli that may cause inflammatory reactions are applied, expression of pro-inflammatory cytokines such as TNF-α and the like is induced, the produced pro-inflammatory cytokines stimulate expression of genes for coding iNOS and COX-2, and nitric oxide (NO) and PGE₂ substance involved in inflammatory reactions are produced so as to cause inflammatory reactions. The nitric oxide can be produced by stimulating iNOS with IFN-γ, STAT-1, NF-κB, GSK-3β, and c-Raf.

Therefore, if the pro-inflammatory cytokines and the inflammation-inducing substances such as nitric oxide are excessively secreted or the cells are kept in an activated state for a long time, a serious side effect such as tissue damage can be caused.

Accordingly, it was found that the veratric acid of the present invention can inhibit expression, activities, and the like of the above-described inflammation-inducing substances in the LPS-stimulated macrophage (RAW264.7) cells, and thus, it can be used as an anti-inflammatory drug.

Further, the present inventors established that inhibition of production of pro-inflammatory cytokines by the veratric acid of the present invention was caused by inhibition of activities of NF-κB and MAPK as their superordinate signaling systems.

Typically, when signal transduction of the NF-κB and MAPK is activated, inflammation-inducing factors are produced and production thereof is promoted. Therefore, if signal transduction of the NF-κB and MAPK is inhibited, production of inflammation-inducing physiological active substances can be inhibited and eventually, inflammatory diseases can be prevented and treated.

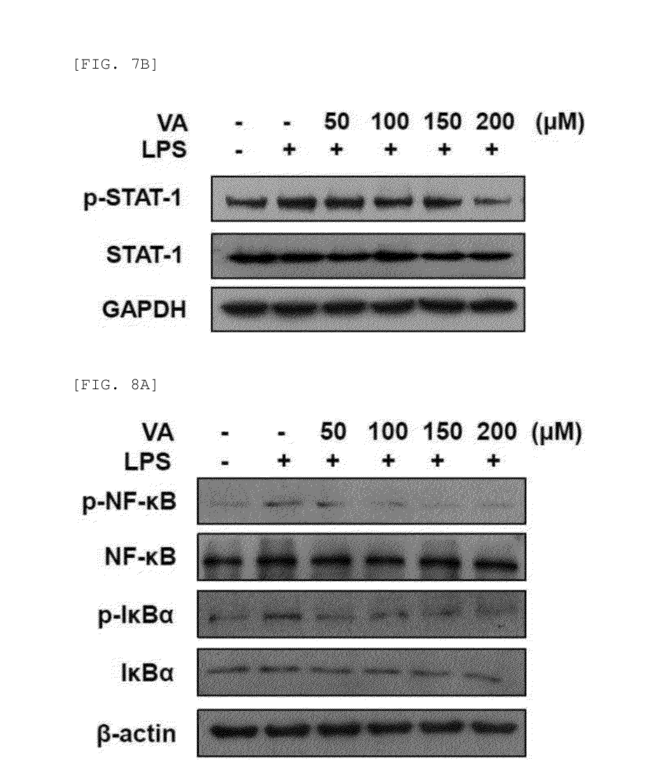

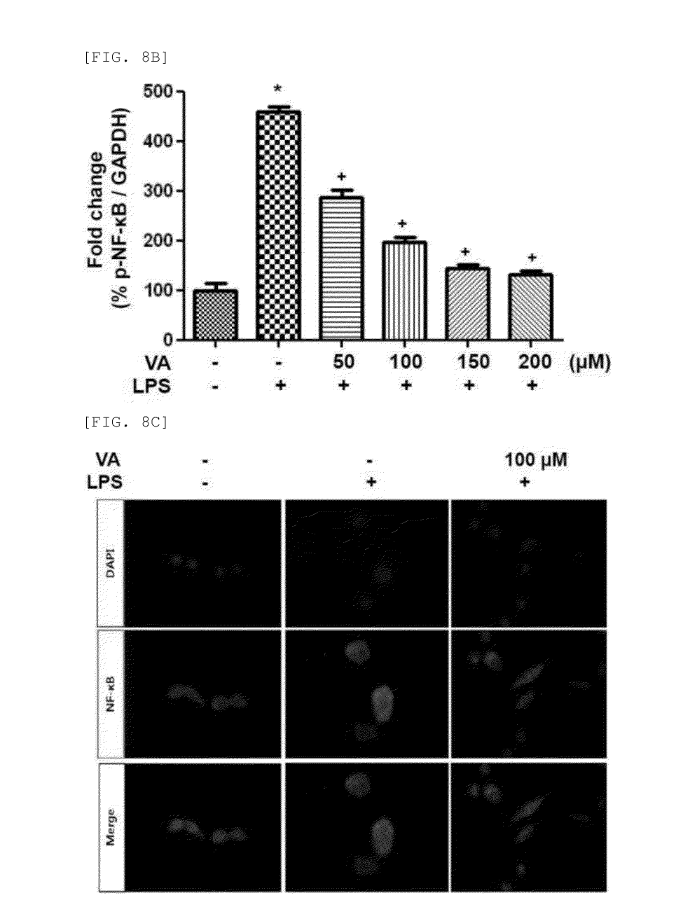

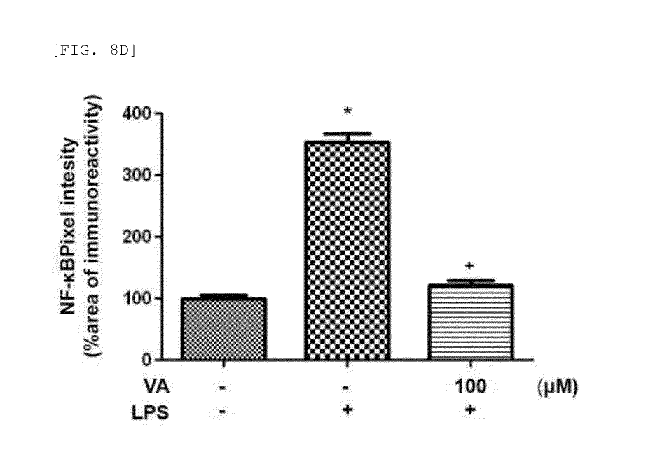

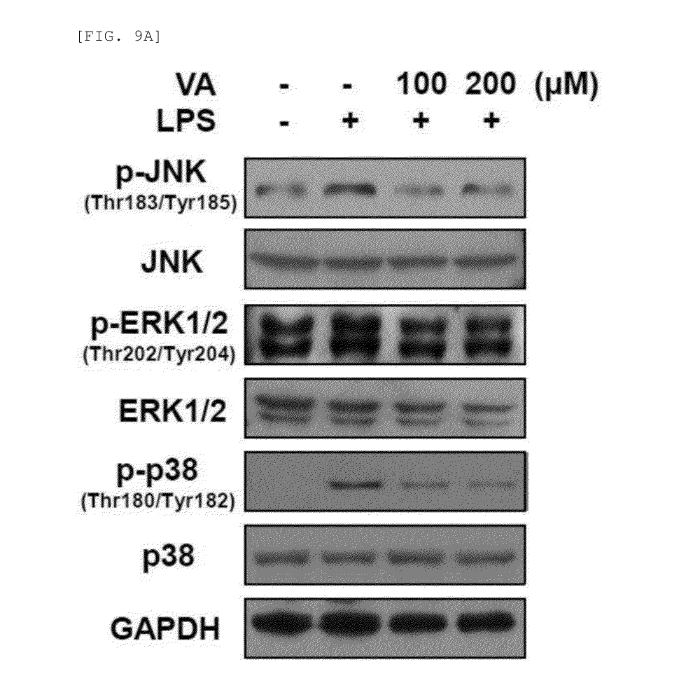

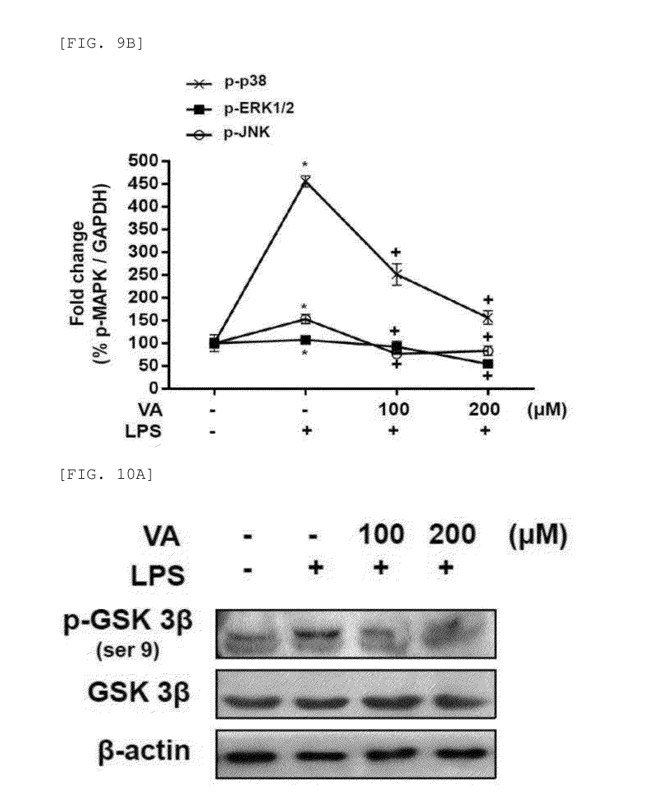

In an example of the present invention, inflammation was caused by LPS in veratric acid-treated macrophage (RAW264.7) cells and veratric acid-untreated macrophage (RAW264.7) cells, and then an activity of NF-κB and phosphorylation of MAPK were measured. In the veratric acid-treated group, it was observed that activities of the NF-κB and MAPK were maintained at a level similar to the control group (see FIGS. 8 and 9) and also, it was observed by using an immunofluorescent microscope that a transcriptional activity of the NF-κB was effectively inhibited (see FIGS. 8C and 8D).

It is known that a nuclear factor kappa B (NF-κB) as a transcription factor is activated by various external stimuli such as inflammation-inducing cytokines, toxic compounds, bacterial infections, viral infections, radiation, UV, oxygen free radicals, and the like and regulates protein expression involved in various cellular reactions such as apoptosis, immune reactions, inflammatory reactions, and the like. Therefore, abnormal activities of the NF-κB are involved in various cancers, inflammatory diseases such as and arthritis, allergic diseases such as asthma or atopic dermatitis, and inflammatory diseases. Thus, a substance of inhibiting the activities of the NF-κB has been a target of a medicine for treating the above-described diseases [A. S. Baldwin Jr, J. Clin. Invest., 2001, 107, 241-246].

When the NF-κB is in a normal state, it exists in an intracellular cytosol. However, if the NF-κB is stimulated by free radicals, inflammation-inducing stimuli, carcinogens, toxins, UV, and the like, it becomes activated and moves into a nucleus. Within the nucleus, the NF-κB induces expressions of various genes that cause inhibition of apoptosis, cell modification, proliferation, infiltration, metastasis or chemical resistance, inflammation, and the like [Chen., Biochem Biophy Res Comm, 2005, 332:1; Jove et al., Endocrinology, 2005, 146:3087; Dandrona et al., Circulation, 2005, 111:1448; Aggarwal et al., Ann N Y Acad Sci, 2004, 1030:434]. Therefore, if the activities of the NF-κB are inhibited, it is possible to inhibit expressions of genes relating to the above-described diseases.

In the present invention, it was observed from phosphorylation of the NF-κB and IκBα that when there was no external stimulus, the NF-κB formed a complex with an inhibiting protein such as inhibitory-KB (IκB) within cytoplasm, and when an extracellular stimulus was applied, the IκB was inactivated and decomposed by phosphorylation, and the NF-κB was dissociated from the IκB and moved into a nucleus and became activated. As a result, the NF-κB enables iNOS and COX-2 to be expressed, and nitric oxide and PGE₂ were produced in excessive amounts so as to cause inflammation.

Further, the veratric acid of the present invention can inhibit activities of members of the MAPK family. The MAPK is well known as a representative signal transduction pathway through which an extracellular stimulus is transmitted from a cell membrane to an intracellular nucleus. The MAPK transmits activated signals from a growth hormone receptor, a cytokine receptor, and a stress receptor into a cell so as to be involved in proliferation, differentiation, and apoptosis of a cell. The MAPK can be roughly classified into 1) an extracellular signal-activated kinase (ERK), 2) a c-JUN N-terminal kinase (JNK), and 3) p38 MAPK. The ERK (ERK1/2) is mainly involved in signal transduction of a growth hormone and plays a key role in proliferation and differentiation of a cell. Meanwhile, it is known that the p38 MAPK and the JNK classified as stress kinases are activated by extracellular stress stimuli and mediate in inflammatory reactions, immune reactions, and cell apoptosis. Therefore, it is possible to treat inflammatory diseases by using these MAP kinase inhibitors.

Accordingly, based on the above descriptions, the present invention can provide a pharmaceutical composition comprising a veratric acid as an effective component for preventing and treating inflammatory diseases.

In the present invention, the “inflammatory diseases” include diseases with inflammation induced by various stimulation factors, such as NO, iNOS, COX-2, PGE₂, and TNF-α, which cause a series of inflammatory reactions. The inflammatory diseases include, but is not limited to, typical inflammatory symptoms such as edema, inflammatory bowel disease, peritonitis, osteomyelitis, cellulites, pancreatitis, trauma-induced shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, infectious arthropathy, post-infectious arthritis, gonococcal arthritis, tuberculous arthritis, viral arthritis, fungal arthritis, syphilitic arthritis, Lyme disease, arthritis related to “vasculitis syndrome”, polyarteritis nodosa, hypersensitivity vasculitis, Lou Gehrig's granulomatosis, polymyalgia rheumatica, joint cell arteritis, calcium pyrophosphate deposition arthropathy, pseudogout, non-articular rheumatism, bursitis, tendovaginitis, epicondylitis (tennis elbow), neuropathic joint disease (or referred to as “charcot joint”), hemarthrosis, Henoch-Schδnlein purpura, hypertrophic osteoarthropathy, multicentric reticulohistiocytoma, scoliosis, hemochromatosis, hemoglobinopathy, hyperlipoproteinemia, hypogammaglobulinemia, familial Mediterranean fever, Behat's disease, systemic lupus erythematosus, relapsing fever, multiple sclerosis, septicemia, septic shock, acute respiratory distress syndrome, multiorgan dysfunction syndrome, chronic obstructive pulmonary disease, rheumatoid arthritis, acute lung injury, and broncho-pulmonary dysplasia. Further, the inflammatory diseases include inflammatory skin diseases such as acute/chronic eczema, contact dermatitis, atopic dermatitis, seborrheic dermatitis, exfoliative dermatitis, solar dermatitis, and psoriasis.

Further, the composition according to the present invention may comprise only a veratric acid in a pharmaceutically effective amount or may comprise one or more pharmaceutically acceptable carriers, excipients, or diluents. The pharmaceutically effective amount means an amount sufficient to prevent, improve, and treat symptoms of the inflammatory diseases. The veratric acid may be contained at a concentration of 0.1 to 200 μM in the composition.

Furthermore, according to the present invention, a pharmaceutically effective amount of the veratric acid is 0.5 to 100 mg/day/weight kg and preferably 0.5 to 10 mg/day/weight kg. However, the pharmaceutically effective amount can be appropriately changed depending on a degree of symptoms of inflammatory or immune diseases, an age of a patient, a weight of a patient, a health condition of a patient, a sex of a patient, an administering route, a period of treatment, and the like.

The pharmaceutically acceptable composition means that it is physiologically acceptable and an allergic reaction or the similar reaction thereof, such as a gastroenteric trouble, and dizziness, is not caused typically when being administered to humans. Examples of the carrier, excipient, and diluents may include lactose, dextrose, sucrose, sorbitol, mannitol, xylitol, erythritol, maltitol, starch, acacia rubber, alginate, gelatin, calcium phosphate, calcium silicate, cellulose, methylcellulose, polyvinylpyrrolidone, water, methyl hydroxybenzoate, propyl hydroxybenzoate, talc, magnesium stearate, and minerals. Further, they may additionally include fillers, anticoagulants, lubricants, wetting agents, flavoring, emulsifying agents, preservatives, and the like.

Further, the composition of the present invention may be formulated by using a method publicly known in the art in order to provide rapid, continuous, or delayed release of an effective component after being administered to mammals. A dosage form may be powder, granule, tablet, emulsion, syrup, aerosol, soft or hard gelatin capsule, a sterile injection solution, or a sterile powder.

The composition according to the present invention can be administered in various ways for example, orally, percutaneously, subcutaneously, intravenously or intramuscularly. A dosage of the effective component may be selected appropriately depending on various factors, such as an administering route, an age, a sex, and a weight of a patient, severity of a patient, and the like. Further, the composition for preventing and improving symptoms of inflammatory diseases of the present invention may be administered along with a compound publicly known as having an effect of preventing, improving, or treating symptoms of inflammatory or immune diseases.

Therefore, the present invention can provide a drug comprising a veratric acid for preventing and treating symptoms of inflammatory diseases and particularly can provide an anti-inflammatory drug comprising the composition.

Moreover, the veratric acid according to the present invention does not induce toxicity to cells and does not cause side effects, and thus, it can be used safely in the body. Accordingly, the veratric acid can be used as a food composition for preventing and improving inflammatory diseases.

Therefore, the food composition comprising the veratric acid as an effective component for preventing and improving inflammatory diseases can be used easily for foods effective in preventing and improving symptoms of inflammatory diseases, such as main materials or added materials of foods, food additives, functional foods or beverages.

According to the present invention, the term “food” means a natural substance or a processed material that may include one or more nutrients and preferably to be edible after some processes. Typically, it includes all of foods, food additives, health functional foods, and beverages.

The foods that can contain the composition for preventing and improving symptoms of inflammatory diseases according to the present invention may include, for example, all sorts of foods, beverages, gums, teas, vitamin complexes, health functional foods, and the like. Additionally, the foods of the present invention may be special nutritious foods (for example, milk formulas, young children and baby foods, etc.), processed meat products, fish meat products, bean cured foods, jellied foods, noodles (for example, ramens, noodles, etc.), breads, dietary supplements, seasoning foods (for example, soy sauce, soybean paste, red pepper paste, mixed soy paste, etc.), sauces, confectionery (for example, snacks), candies, chocolates, gums, ice creams, milk products (for example, fermented milk, cheese, etc.), other processed foods, Kimchi, salt-fermented foods (all sorts of Kimchis, pickled vegetables, etc.), beverages (for example, fruit beverages, vegetable beverages, soybean milk, fermented drinks, etc.), natural seasonings (for example, ramen soup base powder, etc.), and the like, but the present invention is not limited thereto. The above-described foods, beverages, or food additives can be produced by using a typical production method.

Further, the term “health functional food” refers to a food group added with value such that a function of the food can act or can be expressed for a particular purpose by using physical, biochemical, and biotechnological methods, and the like and also a processed food designed to sufficiently express a body modulating function of the food related to biological defensive rhythm control, prevention of and recovery from diseases, and the like. To be specific, it may be a health functional food. The health functional food may include sitologically acceptable food supplementary additives and may further include appropriate carriers, excipients, and diluents typically used for preparing a health functional food.

Further, the term “beverage” refers to a generic term for drinks for quenching thirst or enjoying taste, and may include a functional beverage. The beverage comprises, as an essential component, the composition for preventing and improving symptoms of inflammatory and immune disease at a directed rate and may comprise other components without any particular limitation and also may include various flavoring agents, natural carbohydrates, and the like as additional components like other typical beverage.

In addition to the above-described components, the food comprising the composition for preventing and improving symptoms of inflammatory disease according to the present invention may include various nutritional supplements, vitamins, minerals (electrolyte), flavoring agents, such as synthetic flavoring agents, natural flavoring agents, etc., colorings, fillers (cheese, chocolates, etc.), a pectic acid and salts thereof, an alginic acid and salts thereof, an organic acid, protective colloid thickeners, pH control agents, stabilizers, preservatives, glycerin, alcohol, and carbonating agents used for carbonated drinks, etc. The above-described components may be used alone or in combination.

In the food including the composition for preventing and improving symptoms of inflammatory disease of the present invention, the composition of the present invention may be included in the amount of 0.001 wt % to 90 wt o, and preferably, 0.1 wt % to 40 wt %. As for the beverage, it may be included in the amount of 0.001 g to 2 g, and preferably 0.01 g to 1 g based on 100 ml. However, when the composition is taken for a long time for improving health and hygiene or for managing health, it may be included in the amount equal to or less than the above ranges. Since an effective component has no problem in terms of safety, it may be used in the amount equal to or greater than the above ranges. Therefore, the present invention is not limited thereto.

Hereinafter, the present invention will be described in more detail with reference to Examples. However, Examples will be provided only for illustrating the present invention. Thus, it would be obvious to those skilled in the art that the scope of the present invention is not limited to Examples.

### Example 1

### Evaluation of Cytotoxicity of Veratric Acid (3,4-Dimethoxybenzoic acid)

Cell lines used to evaluate cytotoxicity of a veratric acid were macrophage RAW264.7, human embryonic kidney (HEK) 293, and human keratinocyte (HaCaT) cells. For a cytotoxicity experiment, the cell lines were first incubated in a culture solution of DMEM containing 10% fetal bovine serum (FBS) and 1% penicillin (100 U/ml)/streptomycin (100 U/ml) under a condition of 5% CO₂ at a temperature of 37° C. Then, the incubated cells were seeded in a 96-well plate at 1×10⁵ cells/well. Each of the cells was treated at different concentrations (0, 50, 100, 150 and 200 μM) of a veratric acid and incubated for 3 hours. After the incubation, the cells were stimulated with 100 ng/ml LPS contained in a culture medium for 24 hours. Thereafter, the culture solution was removed, and then, the cells were treated with a solution of WST-1® (Daeil Lab service, Korea) and further incubated for 3 hours. An optical density of the culture solution was measured at 460 nm by using an ELIZA analyzer to analyze cell viability.

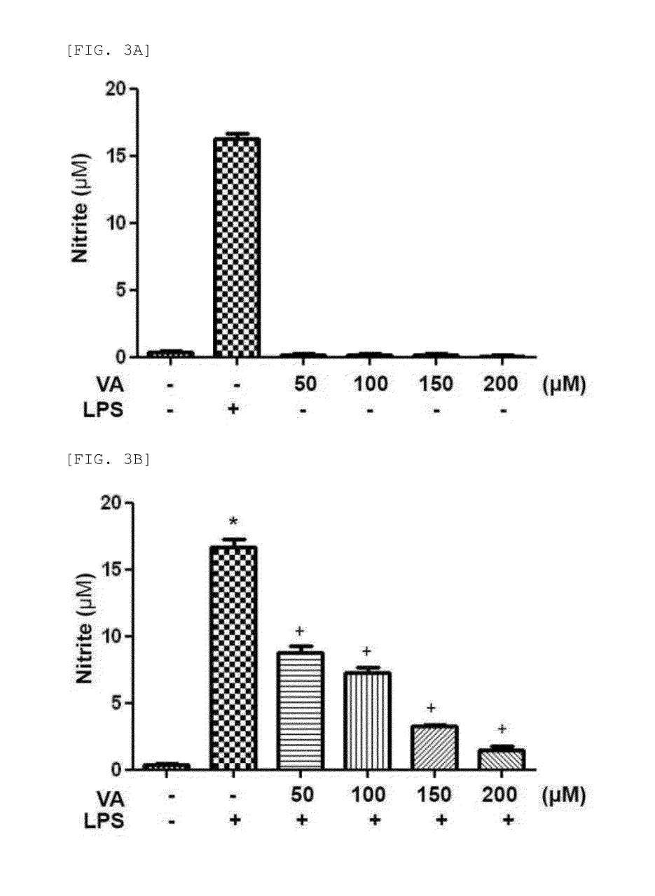

As shown in FIG. 2, a result thereof indicated that each cell treated at different concentrations of a veratric acid was not much different in cell viability from a non-treated control group, and the veratric acid did not show cytotoxicity to all of the cells at a high concentration of 200 μM. Therefore, based on this result, the present inventors found that the veratric acid did not cause toxicity to the cells and thus was stable in the body.

### Example 2

### Analysis of Veratric Acid (3,4-Dimethoxybenzoic Acid)'s Activity of Inhibiting Nitric Oxide (NO) Production

In order to confirm whether or not the veratric acid has an activity of inhibiting production of nitric oxide that causes inflammation, the following experiment was performed. The prepared macrophage RAW264.7 cells were seeded in a 96-well plate at 1×10⁵ cells/well. Each of the cells was treated at different concentrations (0, 50, 100, 150 and 200 μM) of the veratric acid and incubated for 3 hours. The cells were incubated in a culture medium containing 100 ng/ml LPS for 24 hours. Thereafter, a culture solution was acquired. After the culture solution was allowed to react with a Griess reagent, a production level of nitric oxide induced by the LPS was measured at 540 nm by using the ELIZA analyzer (BD PharMingen, USA).

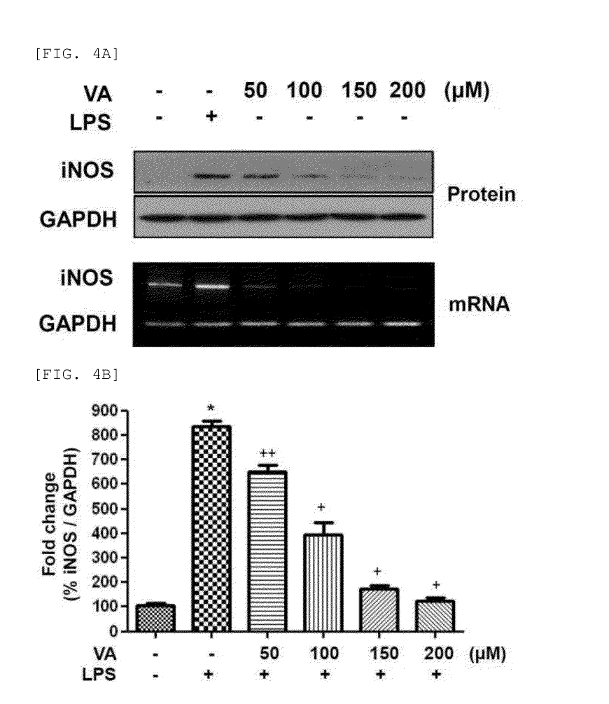

As shown in FIG. 3A, it could be seen from a result thereof that when the macrophage cells were treated with the LPS and the veratric acid independently, the macrophage cells were stimulated by the LPS and the amount of nitric oxide produced was increased, whereas when the macrophage cells were treated with the veratric acid only, the amount of nitric oxide produced was similar to that of a control group or nitric oxide was rarely produced. Further, as shown in FIG. 3B, it could be seen that when the macrophage cells were treated with the LPS in combination with the veratric acid, excessive production of nitric oxide in the macrophage cells stimulated by the LPS was inhibited by the veratric acid and a level of inhibition of nitric oxide production was in proportion to a concentration of the veratric acid used.

### Example 3

### Analysis of Effect of Veratric Acid (3,4-Dimethoxybenzoic Acid) on Inducible Nitric Oxide Synthase (iNOS)

From Example 2, it was found that the veratric acid inhibited production of nitric oxide in the LPS-stimulated macrophage cells. Thus, the present inventors performed an RT-PCR and a Western blot analysis as follows in order to confirm whether or not the veratric acid's mechanism of inhibiting nitric oxide production was operated by regulating expression and protein activity of iNOS as an inducible nitric oxide synthase involved in nitric oxide production.

(1) RT-PCR

The prepared murine macrophage RAW264.7 cells treated with the LPS in combination with the veratric acid were used. RNA was extracted and separated from the incubated cells by using 2-mercaptoethanol (Sigma, USA). An RT-PCR was performed by using primers listed in Table 1 with the extracted RNA as a template. Then, after electrophoresis, an expression level of a gene was checked with ultraviolet light.

TABLE 1
Primer sequence

| Gene | Sequence (5′->3′) | |
|---|---|---|
| name | Sense (forward) | Antisense (reverse) |
| iNOS | ATG-TCC-GAA-GCA-AAC- | TAA-TGT-CCA-GGA-AGT- |
| | ATC-AC | AGG-TG |
| | (Sequence ID. No. 1) | (Sequence ID. No. 2) |
| GAPDH | AGG-CCG-GTG-CTG-AGT- | TGC-CTG-CTT-CAC-CAC- |
| | ATG-TC | CTT-CT |
| | (Sequence ID. No. 3) | (Sequence ID. No. 4) |

(2) Western Blot

The prepared murine macrophage cells treated with the LPS in combination with the veratric acid were collected and lysed by using a lysis buffer {50 mM Tris-Cl [pH7.5], 150 mM NaCl, 1 mM DTT, 0.5% NP-40, 1% Triton X-100, 1% deoxycholate, 0.1% SDS and a cocktail of proteinase inhibitors [1 mM PMSF, 1 mM EDTA, 11 μM aprotinin, 11 μM leupeptin, and 11 μM pepstatin A (Intron Biotechnology, Gyeonggi, Korea)]}. Protein samples were electrophoresed by 12% SDS-PAGE and then electrically transferred to a nitrocellulose membrane. Thereafter, the protein samples were allowed to react with primary antibodies capable of recognizing the iNOS. After the membrane was washed and then allowed to react with horseradish peroxidase-linked anti-rabbit IgG or anti-rabbit IgG (Cell Signaling Technology Inc., USA). Then, signals were checked by using an enhanced chemiluminescent (ECL) detection system (Pierce, USA).

As shown in FIG. 4, it could be seen from a result thereof that when the murine macrophage cells were treated with the veratric acid, expression and protein activity of the iNOS was decreased or inhibited strongly depending on a concentration of the veratric acid. Therefore, based on this result, the present inventors found that the veratric acid inhibited expression of the iNOS in the LPS-stimulated macrophage cells so as to remarkably reduce a protein expression level.

### Example 4

### Analysis of Effect of Veratric Acid (3,4-Dimethoxybenzoic Acid) on Gene and Protein Expression of Cyclooxygenase-2 (COX-2)

The present inventors performed an RT-PCR and a Western blot analysis in the same manner as Example 3 in order to confirm an effect of the veratric acid on gene and protein expression of cyclooxygenase-2 (COX-2) capable of catalyzing production of prostaglandin G₂ (PGE₂) known as causing an inflammatory reaction. Sequences of primers used for the RT-PCR are shown in Table 2.

TABLE 2
Primer sequence

| Gene | Sequence(5′->3′) | |
|---|---|---|
| name | Sense (forward) | Antisense (reverse) |
| COX-2 | GGG-GTA-CCT-TCC-AGC- | GAA-GAT-CTC-GCC-AGG- |
| | TGT-CAA-AAT-CTC | TAC-TCA-CCT-G |
| | (Sequence ID. No. 5) | (Sequence ID. No. 6) |
| GAPDH | AGG-CCG-GTG-CTG-AGT- | TGC-CTG-CTT-CAC-CAC- |
| | ATG-TC | CTT-CT |
| | (Sequence ID. No. 3) | (Sequence ID. No. 4) |

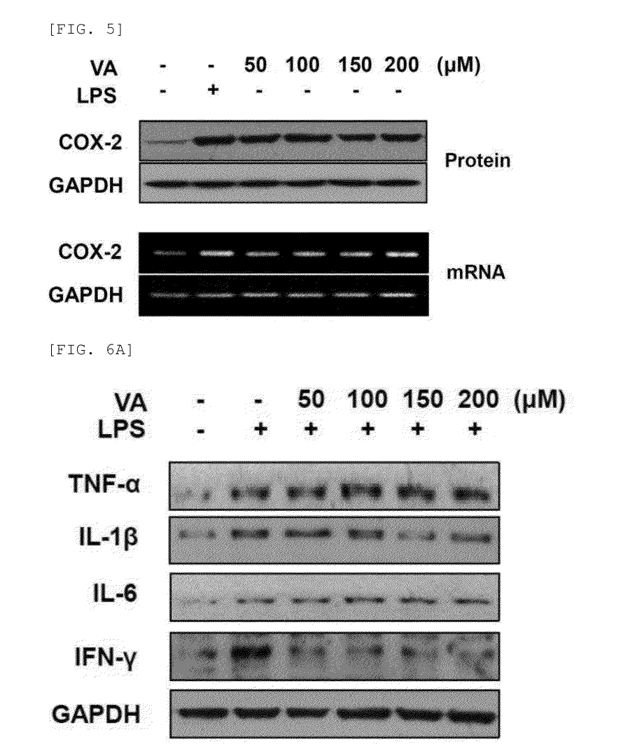

As shown in FIG. 5, a result thereof indicated that the veratric acid did not affect gene and protein expression of COX-2.

### Example 5

### Analysis of Effect of Veratric Acid (3,4-Dimethoxybenzoic Acid) on Protein Expression of Pro-Inflammatory Cytokine and Interferon-γ (IFN-γ)

From the results of the above Examples, it was confirmed that the veratric acid of the present invention inhibited and reduced production of nitric oxide caused by stimulation of the LPS, which resulted from the mechanism of inhibiting expression of the iNOS. Thus, the present inventors checked protein expression levels of IL-1β, IL-6, TNF-α, and IFN-γ through a Western blot analysis performed in the same manner as Example 3 in order to confirm whether or not an anti-inflammatory activity of the veratric acid inhibited production of pro-inflammatory cytokines that cause inflammation and also inhibited secretion of IFN-γ caused by the cytokines.

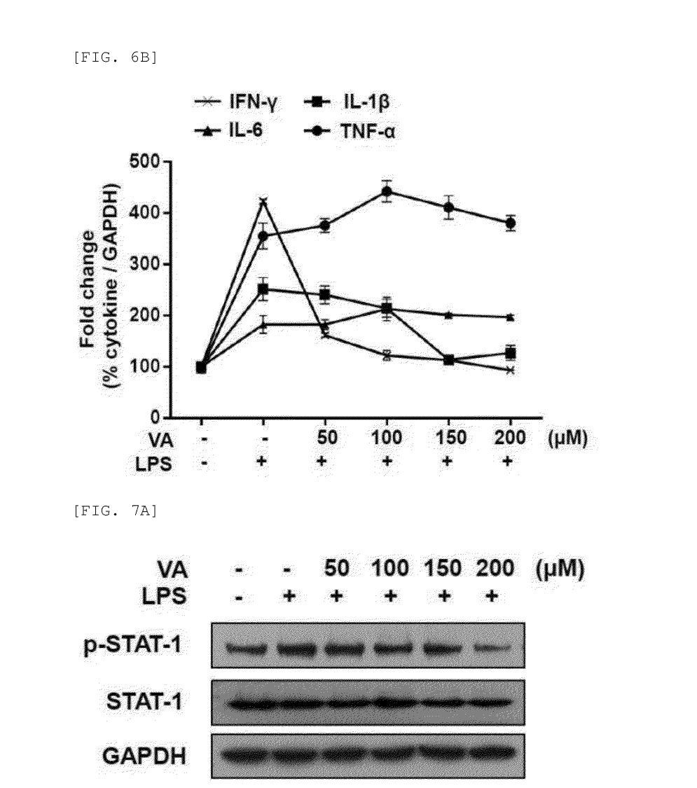

As shown in FIG. 6, a result thereof indicated that in the LPS-stimulated murine macrophage RAW264.7 cells, the veratric acid did not affect protein expression levels of IL-6 and TNF-α but reduced a protein expression level of IL-1β. Further, the veratric acid did not much affect the cytokines but reduced a protein expression level of IFN-γ.

It could be seen from this result that at the time of production of the pro-inflammatory cytokines caused by the LPS as an inflammatory-inducing factor, the veratric acid specifically inhibited the IL-1β and effectively reduced or inhibited the IFN-γ.

### Example 6

### Analysis of Effect of Veratric Acid (3,4-Dimethoxybenzoic Acid) on Phosphorylation of STAT-1

It is known that secreted IFN-γ promotes phosphorylation of STAT-1 and induces transcription of iNOS, and an activity of the iNOS causes production of nitric oxide and induces inflammation. Therefore, protein expression levels of STAT-1 and phosphorylated STAT-1 were checked through a Western blot analysis performed in the same manner as Example 3 in order to confirm an effect of the veratric acid, which reduced the protein level of the IFN-γ in Example 5, on phosphorylation of STAT-1.

As shown in FIG. 7, a result thereof indicated that the veratric acid, which reduced the IFN-γ in the LPS-stimulated macrophage cells, reduced phosphorylation of the STAT-1 depending on a concentration of the veratric acid. Based on this result, the present inventors found that the veratric acid of the present invention inhibited protein expression levels of the IL-1β and the IFN-γ under a LPS-stimulated inflammation-inducing environment, resulting in reduction of phosphorylation of the STAT-1 and reduction of transcription of the iNOS so as to reduce production of nitric oxide.

### Example 7

### Effect of Veratric Acid (3,4-Dimethoxybenzoic Acid) on Phosphorylation of NF-κB and IκBα

An NF-κB is a transcription factor and when the NF-κB is activated by phosphorylated IκBα, it is moved into a nucleus and bound to a promoter site such that target molecules can be transcribed. Thus, the present inventors performed a Western blot analysis and an immunofluorescent microscopy as follows in order to confirm whether or not the veratric acid affected protein levels of the NF-κB and the IκBα. That is, protein expression levels of the NF-κB and the IκBα in the LPS-stimulated macrophage cells were checked through the Western blot analysis performed in the same manner as Example 3, and in the immunofluorescent microscopy, after 100 μM veratric acid was treated for 1 hour, LPS was incubated for 12 hours, and 4,6-diamidino-2-phenylindole (DAPI) was treated at room temperature for 15 minutes, the cells were immobilized with 4% formaldehyde and then distribution of the NF-κB in the cells was checked visually.

As shown in FIG. 8, a result thereof indicated that when the veratric acid was treated, the protein expression of the phosphorylated NF-κB was remarkably reduced and a level of reduction was in proportion to a concentration of the veratric acid used. Further, the phosphorylated IκBα was also reduced by the veratric acid (see FIGS. 8A and 8B). A result of checking a distribution pattern of the NF-κB in the cells indicated that the veratric acid inhibited movement of the NF-κB into the nucleus (see FIGS. 8C and 8D). Therefore, it was found that the veratric acid used in the present invention reduced phosphorylation of the IκBα so as to inhibit movement of the NF-κB into the nucleus from protoplasm and inhibited phosphorylation of the NF-κB and the IκBα so as to regulate expression of the iNOS.

### Example 8

### Effect of Veratric Acid (3,4-Dimethoxybenzoic Acid) on Phosphorylation of MAPK

Protein expression levels of JNK, p38, and ERK1/2 in the LPS-stimulated macrophage cells were checked through a Western blot analysis performed in the same manner as Example 3 in order to confirm whether or not the veratric acid affected an activity of MAPK as a superordinate signaling system that produced pro-inflammatory cytokines.

As shown in FIG. 9, a result of the Western blot analysis indicated that when the veratric acid was treated, phosphorylation of the JNK, the p38, and the ERK1/2 was inhibited, and the veratric acid had a stronger effect on regulation of phosphorylation of the p38 (see FIG. 9B).

### Example 9

### Effect of Veratric Acid (3,4-Dimethoxybenzoic Acid) on Phosphorylation of GSK-3β

A protein expression level of GSK-3β in the LPS-stimulated macrophage cells was checked through a Western blot analysis performed in the same manner as Example 3 in order to confirm whether or not the veratric acid affected phosphorylation of the GSK-3β as a superordinate signaling system that caused inflammatory reactions.

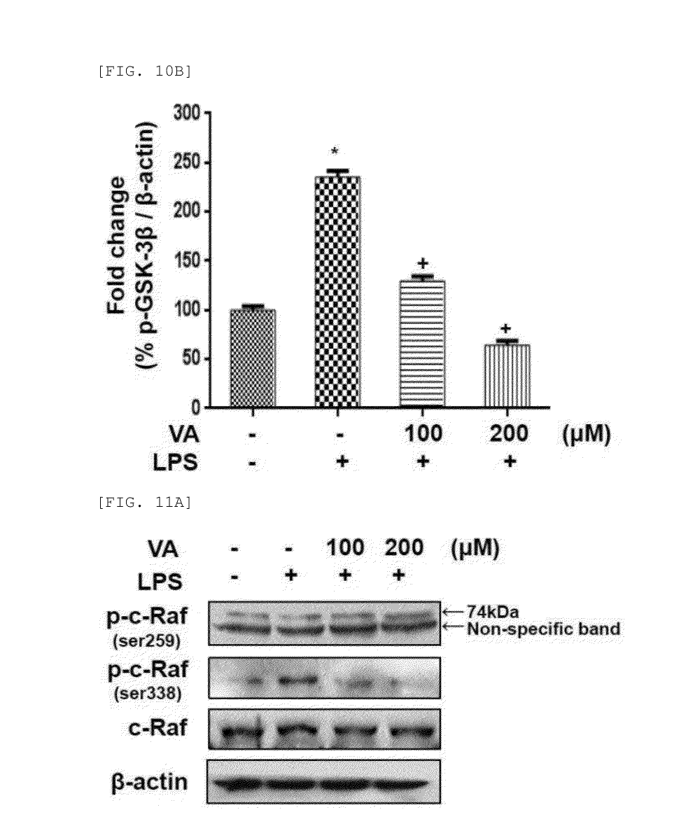

As shown in FIG. 10, a result thereof indicated that when the veratric acid was treated, the veratric acid remarkably inhibited phosphorylation of the GSK-3β depending on a concentration of the veratric acid. Based on this result, it is known that inhibition of the GSK-3β in the LPS-stimulated murine macrophage RAW264.7 cells used in the present invention reduced expression of iNOS and increased production of interleukin-1β (IL-1β).

### Example 10

### Effect of Veratric Acid (3,4-Dimethoxybenzoic Acid) on Phosphorylation of c-Raf

If c-Raf (RAF proto-oncogene serine/threonine-protein kinase) is phosphorylated at Ser³³⁸, the c-Raf induces expression of iNOS. Thus, the present inventors checked a protein level of the c-Raf in the LPS-stimulated macrophage cells and a phosphorylated protein level at Ser²⁵⁹ and Ser³³⁸ of the c-Raf through a Western blot analysis performed in the same manner as Example 3 in order to confirm whether or not the veratric acid affected the protein level of the c-Raf.

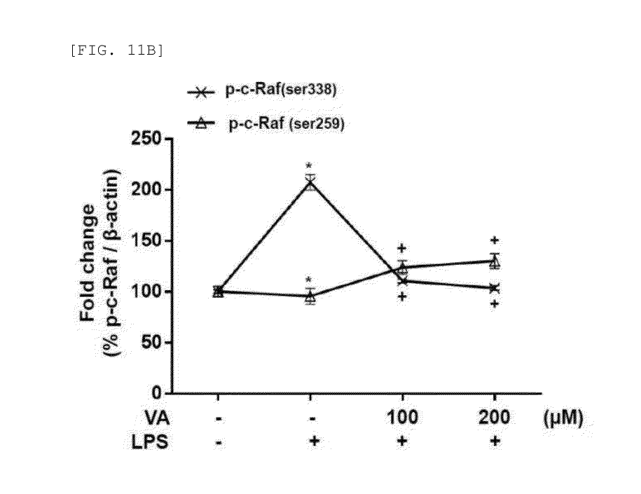

As shown in FIG. 11, a result thereof indicated that when the veratric acid was treated, it did not affect phosphorylation at Ser²⁵⁹ of the c-Raf but inhibited phosphorylation at Ser³³⁸ of the c-Raf. Therefore, it can be seen from this result that the veratric acid has a stronger effect on the phosphorylation at Ser³³⁸ of the c-Raf.

As set forth above, according to exemplary embodiments of the invention, a veratric acid has an excellent activity of inhibiting production of IL-1β as a pro-inflammatory cytokine and an excellent effect of inhibiting production of MAPK and nitric oxide (NO) in a macrophage cell stimulated by a stimulation factor that causes an inflammatory reaction. Thus, it can be used for development of medicines for treating inflammatory diseases and immune diseases which can be caused by excessive inflammatory reactions. Further, the veratric acid is stable in the body. Thus, it can be used as a material of a health functional food.

While the present invention has been shown and described in connection with the exemplary embodiments, it will be apparent to those skilled in the art that modifications and variations can be made without departing from the spirit and scope of the invention as defined by the appended claims.

What is claimed is:

1. A pharmaceutical composition for preventing or treating an inflammatory disease comprising a veratric acid represented by the following Chemical Formula 1:

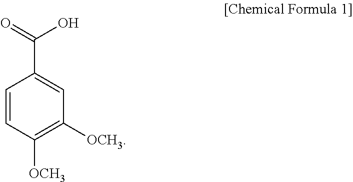 [SMILES: COc1ccc(C(=O)O)cc1OC]

2. The pharmaceutical composition of claim 1, wherein the veratric acid inhibits production or expression of nitric oxide, inducible nitric oxide synthase (iNOS), and interferon-γ (INF-γ); and reduces or inhibits phosphorylation of NF-κB, MAP Kinase (MAPK), STAT-1, GSK-3β, and c-Raf.

3. The pharmaceutical composition of claim 1, wherein the veratric acid is contained at a concentration of 0.1 to 200 μM in the composition.

4. The pharmaceutical composition of claim 1, wherein the inflammatory disease is any one selected from the group consisting of inflammatory bowel disease, peritonitis, osteomyelitis, cellulites, pancreatitis, trauma-induced shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, infectious arthropathy, post-infectious arthritis, gonococcal arthritis, tuberculous arthritis, viral arthritis, fungal arthritis, syphilitic arthritis, Lyme disease, arthritis related to “vasculitis syndrome”, polyarteritis nodosa, hypersensitivity vasculitis, Lou Gehrig's granulomatosis, polymyalgia rheumatica, joint cell arteritis, calcium pyrophosphate deposition arthropathy, pseudogout, non-articular rheumatism, bursitis, tendovaginitis, epicondylitis (tennis elbow), neuropathic joint disease, hemarthrosis, Henoch-Schδnlein purpura, hypertrophic osteoarthropathy, multicentric reticulohistiocytoma, scoliosis, hemochromatosis, hemoglobinopathy, hyperlipoproteinemia, hypogammaglobulinemia, familial Mediterranean fever, Behat's disease, systemic lupus erythematosus, relapsing fever, multiple sclerosis, septicemia, septic shock, acute respiratory distress syndrome, multiorgan dysfunction syndrome, chronic obstructive pulmonary disease, rheumatoid arthritis, acute lung injury, broncho-pulmonary dysplasia, and inflammatory skin disease.

5. An anti-inflammatory drug comprising the pharmaceutical composition for preventing or treating an inflammatory disease of claim 1.

6. A method for preventing or treating an inflammatory disease comprising administering a therapeutically effective amount of a veratric acid represented by the following Chemical Formula 1 to a subject:

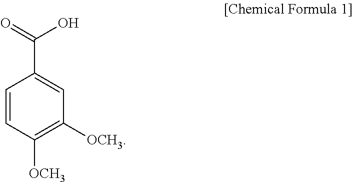 [SMILES: COc1ccc(C(=O)O)cc1OC]

7. The method of claim 6, wherein the inflammatory disease is any one selected from the group consisting of inflammatory bowel disease, peritonitis, osteomyelitis, cellulites, pancreatitis, trauma-induced shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, infectious arthropathy, post-infectious arthritis, gonococcal arthritis, tuberculous arthritis, viral arthritis, fungal arthritis, syphilitic arthritis, Lyme disease, arthritis related to “vasculitis syndrome”, polyarteritis nodosa, hypersensitivity vasculitis, Lou Gehrig's granulomatosis, polymyalgia rheumatica, joint cell arteritis, calcium pyrophosphate deposition arthropathy, pseudogout, non-articular rheumatism, bursitis, tendovaginitis, epicondylitis (tennis elbow), neuropathic joint disease, hemarthrosis, Henoch-Schδnlein purpura, hypertrophic osteoarthropathy, multicentric reticulohistiocytoma, scoliosis, hemochromatosis, hemoglobinopathy, hyperlipoproteinemia, hypogammaglobulinemia, familial Mediterranean fever, Behat's disease, systemic lupus erythematosus, relapsing fever, multiple sclerosis, septicemia, septic shock, acute respiratory distress syndrome, multiorgan dysfunction syndrome, chronic obstructive pulmonary disease, rheumatoid arthritis, acute lung injury, broncho-pulmonary dysplasia, and inflammatory skin disease.

8. A health functional food for preventing or treating an inflammatory disease comprising a veratric acid represented by the following Chemical Formula 1:

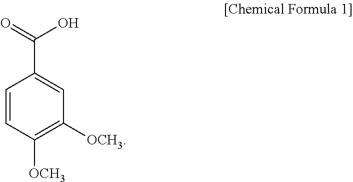 [SMILES: COc1ccc(C(=O)O)cc1OC]

9. The health functional food of claim 8, wherein the veratric acid inhibits production or expression of nitric oxide, inducible nitric oxide synthase (iNOS), and interferon-γ (INF-γ); and reduces or inhibits phosphorylation of NF-κB, MAP Kinase (MAPK), STAT-1, GSK-3β, and c-Raf.

10. The health functional food of claim 8, wherein the inflammatory disease is any one selected from the group consisting of inflammatory bowel disease, peritonitis, osteomyelitis, cellulites, pancreatitis, trauma-induced shock, bronchial asthma, allergic rhinitis, cystic fibrosis, acute bronchitis, chronic bronchitis, acute bronchiolitis, chronic bronchiolitis, osteoarthritis, gout, spondyloarthropathy, ankylosing spondylitis, Reiter's syndrome, psoriatic arthropathy, enteropathic spondylitis, juvenile arthropathy, juvenile ankylosing spondylitis, reactive arthropathy, infectious arthropathy, post-infectious arthritis, gonococcal arthritis, tuberculous arthritis, viral arthritis, fungal arthritis, syphilitic arthritis, Lyme disease, arthritis related to “vasculitis syndrome”, polyarteritis nodosa, hypersensitivity vasculitis, Lou Gehrig's granulomatosis, polymyalgia rheumatica, joint cell arteritis, calcium pyrophosphate deposition arthropathy, pseudogout, non-articular rheumatism, bursitis, tendovaginitis, epicondylitis (tennis elbow), neuropathic joint disease, hemarthrosis, Henoch-Schδnlein purpura, hypertrophic osteoarthropathy, multicentric reticulohistiocytoma, scoliosis, hemochromatosis, hemoglobinopathy, hyperlipoproteinemia, hypogammaglobulinemia, familial Mediterranean fever, Behat's disease, systemic lupus erythematosus, relapsing fever, multiple sclerosis, septicemia, septic shock, acute respiratory distress syndrome, multiorgan dysfunction syndrome, chronic obstructive pulmonary disease, rheumatoid arthritis, acute lung injury, broncho-pulmonary dysplasia, and inflammatory skin disease.
